(19) United States
(12) Patent Application Publication
(10) Pub. No.: US 2015/0119796 A1
(43) Pub. Date: Apr. 30, 2015

(54) Anti-Lockup Thread Attachment Mechanism and Method of Use Thereof

(71) Applicant: DC Devices, Inc., Tewksbury, MA (US)

(72) Inventor: Matthew J. Finch, Medford, MA (US)

(73) Assignee: DC Devices, Inc., Tewksbury, MA (US)

(21) Appl. No.: 14/523,926

(22) Filed: Oct. 26, 2014

Related U.S. Application Data
(60) Provisional application No. 61/896,064, filed on Oct. 26, 2013.

(51) Int. Cl. A61M 25/00

(52) U.S. Cl. CPC A61M 25/0082; A61F 2002/9522
USPC 604/57

(57) ABSTRACT

The present teachings provide thread attachment mechanisms each having an internal and an external threaded portion. The internal and external threaded portions are configured to engage with each other to form an attachment. The thread attachment mechanism in the present teachings further include an anti-lockup feature. The present teachings further provide a cardiac implant engaged with a delivery catheter devices through a thread attachment mechanism having an anti-lockup feature of the present teachings. In one embodiment, the anti-lockup feature prevents the implant from being overly tightened to the delivery catheter. In another embodiment, the anti-lockup feature allows the implant to be released from the delivery catheter in a predictable and controlled manlier. Another aspect of the present teachings provides methods of using a threaded attaching mechanism of the present teachings to deliver, deploy, and release a cardiac implant.

### CROSS-REFERENCE TO RELATED APPLICATION

This application claims priority to and the benefit of U.S. Provisional Application No. 61/896,064, entitled “Anti-lockup Thread Attachment Mechanism and Method of Use Thereof,” tiled on Oct. 26, 2013, which is incorporated herein in its entirety by reference.

### FIELD

The present teachings relate generally to a thread attachment mechanism between a delivery catheter and a medical implant. The present teachings further relate to incorporating such thread attachment mechanism for percutaneous delivering, deploying, and releasing a cardiac implant by a delivery catheter. An example of the present teachings relates to an implant attachment mechanism in a delivery system that prevents the implant from over-tightening to the delivery catheter, and provides a predictable easy release of such implant from the delivery catheter.

### BACKGROUND

Modern medical technology has produced a number of medical implants that are designed for being compressed into a small tube or catheter to facilitate their introduction into the vasculature. Many of these implants are expandable for either occluding an aperture in the heart or creating a shunt between heart chambers. For example, a septal occluder can be used to repair a hole in the heart wall, and an atrial shunt device can be used to create a blood conduit between the left atrium and right atrium.

Numerous systems for percutaneous catheter delivery of implants have been devised over the years in order to assist physicians in delivering and positioning implants within the human body in a minimally invasive manner. A classic attachment mechanism between a delivery catheter and an implant is a screw mechanism, wherein the implant is threaded onto the delivery catheter outside of the body. Such an attachment mechanism between a delivery catheter and an implant is often preferred due to its simplicity in design and intuitiveness in operation.

Essentially, a screw mechanism allows an implant and a delivery catheter to be threaded together. The delivery catheter-implant assembly is then percutaneously inserted into a blood vessel, or a delivery sheath. Upon reaching a treatment site, the implant is deployed and secured to the treatment site. The delivery catheter is then unthreaded from the implant, thereby releasing the implant inside the body.

In an ideal scenario, the torque strength of the thread assembly between the delivery catheter and the implant is pre-set during the catheter-implant attachment step. That is, as a clinician tightens the threads between the implant and the catheter, he/she has set the torque. The delivery system is designed to handle such torque during percutaneous release of the implant once it is satisfactorily deployed.

One problem associated with the thread attachment between a catheter and an implant is that as the catheter-implant assembly winding through the tortuous delivery path, the screw mechanism tightens itself. Occasionally, the screw assembly becoming so tight that the implant is stuck on the catheter. To release the implant, a clinician, sometimes, has to employ special maneuvers, which is inconvenient for the clinician and can traumatize the surrounding anatomy.

Thus, a thread attachment mechanism that does not adversely influence the torque strength of the delivery catheter-implant assembly is needed. Specifically, a thread attachment mechanism that allows a designer and clinicians to have full control over the delivery, deployment, and release of a medical implant in a minimal invasive procedure is needed.

### SUMMARY

One aspect of the present teachings provides a thread attachment mechanism. In various embodiments, the thread attachment mechanism includes an external threaded portion and an internal threaded portion. In sonic embodiments, the external threaded portion includes a thread body, an enlarged end portion, an interface, and a first thread, where at least a part of the first thread extends around the thread body in a helical manner. In certain embodiments, the first thread starts from a first end of the thread body and extends to the interface. In certain embodiments, the interface is between the threaded body and the enlarged end portion.

In various embodiments, the internal threaded portion includes a hollow thread body, a second thread, and a surface. In some embodiments, at least a part of the second thread extends in the hollow thread body in a helical manner. In particular embodiments, the thread starts from a first end of the hollow thread body to a second end inside the hollow thread body. In various embodiments, the first and the second thread are configured to engage with each other.

In various embodiments, the thread attachment mechanism includes an anti-lockup feature. For example, the anti-lockup feature includes an interface and a corresponding surface with matching profiles. In some embodiments, the interface is at the end of the enlarged end of the external threaded portion and the corresponding surface is at a first end of the second threaded portion. In certain embodiments, the anti-lockup feature prevents the external threaded portion and the internal threaded portion from being tightened beyond a pre-determined torque strength.

Another aspect of the present teachings relates to uses of the thread attachment mechanism. In various embodiments, the thread attachment mechanism is incorporated into a delivery-medical implant assembly. For example, the medical implant can be used for treating heart diseases. For example, the medical implant can be used for treating gastrointestinal diseases. Thus, according to some embodiments, one of the medical implant and the delivery device includes one of the external threaded portion and the internal threaded portion and the other includes the other threaded portion. In an exemplary use, the external and internal threaded portions engage with each other until a preset torque strength is reached, the medical device is delivered to a treatment location, the external threaded and internal threaded portions disengage to release the medical implant, and the delivery device is retracted out of the body.

### BRIEF DESCRIPTION OF THE DRAWINGS

FIG. 1 illustrates an exemplary cardiac implant being positioned through an aperture between the left and right atria.

FIGS. 2A and 2B illustrate an external threaded portions of two exemplary thread attachment mechanisms.

FIG. 3 illustrates an internal threaded portion of an exemplary thread attachment mechanism.

FIGS. 4A and 4B illustrate two exemplary thread attachment mechanisms in their engaged state.

FIGS. 5A and 5B illustrate an exemplary traditional attachment mechanism in their disengaged and engaged states, respectively.

FIG. 6 illustrates torque-angular rotation curves showing the difference between an exemplary traditional threaded assembly and an exemplary anti-lockup threaded assembly according to the present teachings.

FIG. 7 illustrates an exemplary delivery device-implant assembly where the implant is deployed across the atrial septum, and the delivery device and the implant are engaged.

FIG. 8 illustrates an exemplary delivery device-implant assembly where the implant is deployed across the atrial septum, and the delivery device and the implant are disengaged.

### DETAILED DESCRIPTION

Certain specific details are set forth in the following description and Figures to provide an understanding of various embodiments of the present teachings. Those of ordinary skill in the relevant art will understand that they can practice other embodiments of the present teachings without one or more of the details described herein. Thus, it is not the intention of the Applicants to restrict or in any way limit the scope of the appended claims to such details. While various processes are described with reference to steps and sequences in the following disclosure, the steps and sequences of steps should not be taken as required to practice all embodiments of the present teachings. Thus, it is not the intention of the Applicants to restrict or in any way limit the scope of the appended claims to such steps or sequences of steps.

As used herein, the terms “subject” and “patient” refer to an animal, such as a mammal, including livestock, pets, and preferably a human. Specific examples of “subjects” and “patients” include, but are not limited to, individuals requiring medical assistance and, in particular, requiring treatment for symptoms of a heart failure.

As used herein, the term “lumen” means a canal, duct, generally tubular space or cavity in the body of a subject, including veins, arteries, blood vessels, capillaries, intestines, and the like. The term “lumen” can also refer to a tubular space in a catheter, a sheath, or the like in a device.

As used herein, the term “proximal” means close to the operator (less into the body) and “distal” shall mean away from the operator (further into the body). In positioning a medical device from a downstream access point, “distal” is more upstream and “proximal” is more downstream.

As used herein, the term “catheter” or “sheath” encompasses any conduit, including any hollow instrument, that can be inserted into a patients body to treat diseases, to administer or withdraw fluids or to perform a surgical procedure. The catheters of the present teachings can he placed within the vascular, urological, gastrointestinal, ophthalmic, and other bodily system, and may be inserted into any suitable bodily lumen, cavity, or duct. For example, a catheter or a sheath of the present teachings can be used to penetrate a body tissue or interstitial cavities and/or provide a conduit for injecting a solution or gas. The term “catheter” or “sheath” is also intended to encompass any elongate body capable of serving as a conduit for one or more of the ablation, expandable, or sensing elements. In the context of coaxial instruments, the term “catheter” or “sheath” can encompass either the outer catheter body or sheath or other instruments that can be introduced through such a sheath. The use of the term “catheter” should not be construed as meaning only a single instrument but rather is used to encompass both singular and plural instruments, including coaxial, nested, and other tandem arrangements. Moreover, the terms “sheath” or “catheter” are sometime used interchangeably to describe catheters having at least one lumen through which an instrument or treatment can pass.

Unless otherwise specified, all numbers expressing quantities, measurements, and other properties or parameters used in the specification and claims are to be understood as being modified in all instances by the term “about.” Accordingly, unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

An aspect of the present teachings provides a thread attachment mechanism. In various embodiments, the thread attachment mechanism includes an anti-lockup feature that prevents the engaged internal and external threaded portions from further tightening the thread engagement. According to one embodiment of the present teachings, the anti-lockup feature includes an interface on the external threaded portion and a corresponding surface on the internal threaded portion. For example, in one embodiment, as the threaded portions fully engage with each other, the interface matches the corresponding surface and the anti-lockup feature prevents the threads from further advancing over each other. According to one embodiment of the present teachings, the torque strength needed to disengage the thread assembly is pre-set when the thread attachment mechanism is assembled.

Another aspect of the present teachings provides a thread attachment mechanism which is adapt to join a medical implant with a delivery catheter for percutaneous delivery and deployment at a treatment location inside the body. In various embodiments, such thread attachment mechanism between the delivery catheter and the medical implant further includes an anti-rotation feature which is configured in such a way that stops further tightening of the implant with the delivery catheter after a predetermined number of threads have been engaged. In some embodiments, the anti-rotation feature is configured to stop when a pre-set amount of torque is reached.

Another aspect of the present teachings provides a delivery assembly. In various embodiments, the assembly includes a catheter which is configured to engage a cardiac shunt implant through a thread attachment. In some embodiments, the assembly is then percutaneously delivered to a treatment location inside a heart. In some embodiments, upon the proper deployment of the cardiac implant at the treatment location, the thread attachment between the implant and the catheter is disengaged. Delivery catheter is then retracted outside of the body.

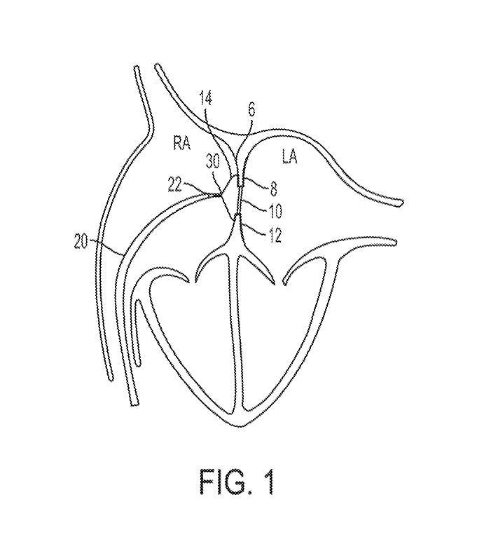

The following description refers to FIGS. 1 to 8. A person with ordinary skill in the art would understand that the figures and description thereto refer to various embodiments of the present teachings and, unless indicated otherwise by their contexts, do not limit the scope of the attached claims.

FIG. 1 illustrates an exemplary cardiac implant (10) being positioned through an aperture (8) between the left and right atria. As used herein, unless otherwise indicated, the term “aperture” includes without being limited to any anatomical anomalies. Examples of an anatomical anomaly include a PFO, an ASD, a VSD, or a shunt otherwise created. According to one embodiment of the present teachings, a delivery sheath is used as a conduit for the percutaneous delivery of a cardiac implant (10). According to one embodiment of the present teachings, the implant (10) is delivered through a standard right heart catheterization procedure. In such a procedure, a cardiac implant (10) is delivered through the femoral vein, through the inferior vena cava, and to the right atrium.

As illustrated in FIG. 1, the exemplary implant (10) is positioned through an aperture (8) across the septum (6). The distal portion (12) of the implant (10) is inside the left atrium. The proximal portion (14) of the implant (10) is inside the right atrium. The proximal end (16) of the implant (10) engages to a distal end (22) of a delivery catheter (20) by a thread attachment mechanism (30) according to one embodiment of the present teachings. A proximal end (not shown) of the delivery catheter (20) remains outside of the body and is controlled by a clinician. In some embodiments, a delivery sheath is also used as a conduit for percutaneously delivering the catheter-implant assembly. In such and other embodiments, a delivery sheath has a distal end, a proximal end, and a longitudinal lumen extending along a longitudinal axis from the proximal end to the distal end. In some embodiments, a delivery catheter (20) is slidably disposed within the longitudinal lumen of the delivery sheath. In other embodiments, the delivery catheter-implant assembly is delivered directly through the blood vessel into the heart without the need of a delivery sheath.

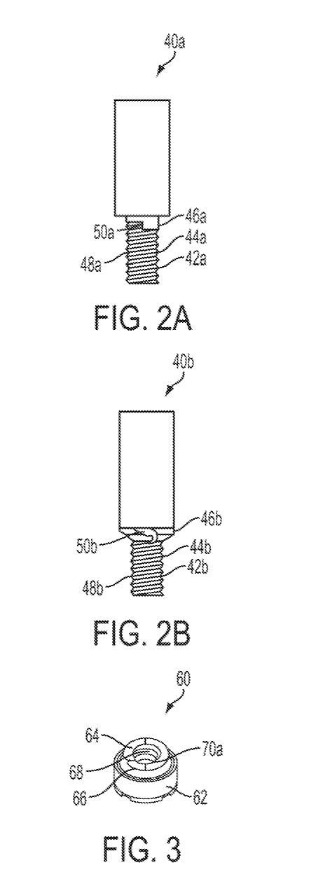

FIGS. 2-4 illustrate exemplary embodiments of the thread attachment mechanism (30) of the present teachings. According to one embodiment, the thread attachment mechanism (30) has a pair of matching external (40) and internal (60) threaded portions. According to one embodiment, one of the external (40) and internal (60) threaded portions could be incorporated at the proximal end of an implant, and the other one of the external (40) and internal (60) threaded portions could be incorporated at the distal end of a delivery catheter. According to one embodiment, the internal threaded portion (60) is at the proximal end of a cardiac implant (10), and the external threaded portion (40) is at the distal end of a delivery catheter (20).

FIG. 2A illustrates an external threaded portion (40a) of an exemplary thread attachment mechanism (30). According to one embodiment, the external threaded portion (40a) includes a thread body (42a) with threads (48a) around the thread body (42a) in the form of a helix. As shown in FIG. 2A, the thread body (42a) of the external threaded portion (40a) includes a cylindrical body portion (44a) with a smaller diameter, and an enlarged end portion (46a) with a diameter greater than the cylindrical body portion (44a). The external threaded portion (40a) of an exemplary thread attachment mechanism (30) further includes an interface (50a). In one embodiment, the interface (50a) is part of the enlarged end portion (46a) as shown in FIG. 2A. In another embodiment, the interface (50a) is on the cylindrical body portion (44a) as a separate structure from the enlarged end portion (46a).

In some embodiments, the thread (48a) starts from a first end of the cylindrical body portion (44a) of the thread body (42a), wraps helically around the cylindrical body portion (44a) toward the enlarged end portion (46a) of the thread body (42a), and ends at the interface (50a) as shown in FIG. 2A. In other embodiments, the thread (48a, 48b) starts from a first location on the cylindrical body portion (44a, 44b) of the thread body (42a, 42b), extends helically around the cylindrical body portion (44a, 44b), and ends at a second location on the cylindrical body portion (44a, 44b). In certain embodiments, the thread (48a, 48b) between the first location and the second location makes at least a quarter of a turn along a helical line. In certain embodiments, the thread (48a, 48b) between the first location and the second location makes at least a half of a turn along a helical line. In certain embodiments, the thread (48a, 48b) between the first location and the second location makes at least a complete turn along a helical line. In certain embodiments, the thread (48a, 48b) between the first location and the second location makes at least one turn along a helical line. In some embodiments, the thread (48a, 48b) of the external threaded portion (40) includes one or more than one threads, each of which is distributed as discussed herein. In certain embodiments, the more than one threads extend along a same helical line. In certain embodiments, the more than one threads extend along two or more helical lines. In specific embodiments, at least two of the two or more helical lines are parallel to one another.

FIG. 2A illustrates one embodiment of the external threaded portion (40a), where the enlarged end portion (46a) is in a generally cylindrical shape. In one embodiment of the present teachings, the enlarged end portion (46a, 46b) of the external threaded portion (40a, 40b) has a general diameter equal or greater than the major diameter of the thread (48). In another embodiment, the enlarged end portion (46b) is in a generally conical shape as shown in FIG. 2B. One skilled in the art should understand that the enlarged end portion can adopt other shapes and forms, especially when various catheter or implant designs are considered. Thus the exemplary embodiments herein should not be construed as limiting to the scope of the present teachings.

In one embodiment, the interface (50a) between the cylindrical body portion (44a) and the enlarged end portion (46a) is a straight surface as shown in FIG. 2A. In another embodiment, the interface (50b) between the cylindrical body portion (44b) and the enlarged end portion (46b) is a curved surface for example as shown in FIG. 2B. One skilled in the art should understand that the interface could have a convex profile, a concave profile, or other curvy profile, for example, in order to satisfy the intended function requirement and/or ease of manufacture.

In one embodiment of the present teachings, the interface (50a, 50b) between the cylindrical body portion (44a, 44b) and the enlarged end portion (46a, 46b) generally aligns with the longitudinal axis of the thread body (42a, 42b), for example, as illustrated in FIG. 2A. In another embodiment of the present teachings, the interface (50a, 50b) between the cylindrical body portion (44a, 44b) and the enlarged end portion (46a, 46b) inclines from the longitudinal axis of the thread body (42a, 42b).

“Angle,” as used herein to describe the relationship between an interface and the longitudinal axis of a thread body, can be described as the angle between the longitudinal axis and an imaginary line obtained by connecting two points in the interface. In one embodiment, at least one angle between the interface (50a, 50b) and the longitudinal axis of the thread body (42a, 42b) ranges from 0° to about 70°. Thus, in some embodiments, at least one angle between the interface (50a, 50b) and the longitudinal axis of the thread body (42a, 42b) ranges from 0° to about 60°, 0° to about 50°, to about 40°, 0° to about 30°, 0° to about 20°, 0° to about 15°, 0° to about 10°, 0° to about 5°. In particular embodiments, at least one angle between the interface (50a, 50b) and the longitudinal axis of the thread body (42a, 42b) ranges from to about 30°, 0° to about 20°, 0° to about 15°, 0° to about 10°, or 0° to about 5°. In particular embodiments, at least one angle between the interface (50a, 50b) and the longitudinal axis of the thread body (42a, 42b) ranges from 0° to about 10°, or 0° to about 5°. In particular embodiments, at least one angle between the interface (50a, 50b) and the longitudinal axis of the thread body (42a, 42b) ranges from 0° to about 10° or 0° to about 5°. In particular embodiments, at least one angle between the interface (50a, 50b) and the longitudinal axis of the thread body (42a, 42b) is about 0°, about 5°, about 10°, about 15°, about 20°, about 25°, about 30°, about 35°, about 40°, about 45°, about 50°, about 55°, about 60°, about 65°, or about 70°.

In one embodiment the thread (48a, 48b) of the external threaded portion (40a, 40b) is a right-handed thread. In another embodiment, the thread (48a, 48b) of the external threaded portion (40a, 40b) is a left-handed thread. In another embodiment, the cross-sectional shape of at least a portion of the thread (48a, 48b), or the thread form is square, triangular, trapezoidal, or other shapes. In one embodiment, the thread angle is 60°. In another embodiment, the thread angle is an angle conventionally used in constructing a thread. In one embodiment, the external threaded portion (40a, 40b) is a single-start. In another embodiment, the external threaded portion (40a, 40b) is a double-start.

FIG. 3 illustrates one embodiment of the internal threaded portion (60) of the thread attachment mechanism (30). According to one embodiment of the present teachings, the internal threaded portion (60) includes a hollow thread body (62) with threads (68) around the inner lumen surface (64) of the thread body (62) in the form of a helix. In other embodiments, the thread (68) starts from a first location along the inner lumen surface (64), extends helically around the inner lumen surface (64), and ends at a second location on the inner lumen surface (64). In certain embodiments, the thread (68) between the first location and the second location makes at least a quarter of a turn along a helical line. In certain embodiments, the thread (68) between the first location and the second location makes at least a half of a turn along a helical line. In certain embodiments, the thread (68) between the first location and the second location makes at least a complete turn along a helical line. In certain embodiments, the thread (68) between the first location and the second location makes at least one turn along a helical line. In some embodiments, the thread (68) of the internal threaded portion (60) include one or more than one threads, each of which is distributed as discussed herein. In certain embodiments, the more than one threads extend along a same helical line. In certain embodiments, the more than one threads extend along two or more helical lines. In specific embodiments, at least two of the two or more helical lines are parallel to one another. According to one embodiment, as the thread (68) winding from outside of the hollow thread body (62) toward the inner lumen surface (64), one end of the thread (68) forms a corresponding surface (70) matching the interface (50a, 50b) of the external threaded portion (40a, 40b).

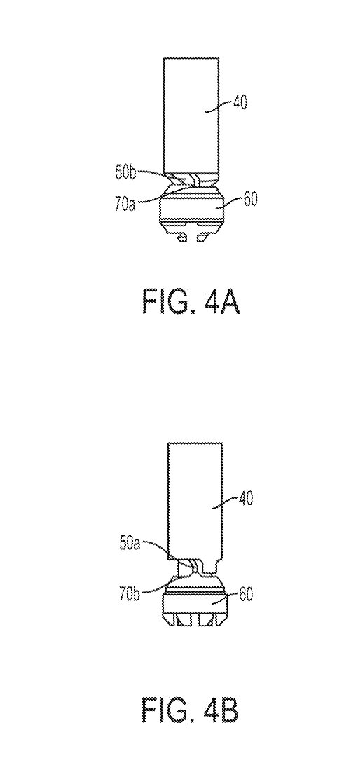

According to one embodiment, the internal threaded portion (60) of the thread attachment mechanism (30) includes a corresponding surface (70a, 70b) that matches the profiles of the interface (50a, 50b) on the external threaded portion (40a, 40b) of the thread attachment mechanism (30). In one embodiment, the corresponding surface (70b) is a structure at one end and on the outside of the lumen of the hollow thread body (62). One exemplary embodiment of such corresponding surface (70b) can be seen in FIG. 4B. Although FIG. 4 illustrates that the corresponding surface (70b) has a triangle cross section, one skilled in the art should understand that the corresponding surface (70b) could have any shape, or form. Thus, the embodiments discussed here should not be construed as limiting.

In another embodiment, the corresponding surface (70a) is part of the thread of the internal threaded portion (60). According to one embodiment, as shown in FIG. 3, the thread (68) on the internal threaded portion (60) starts at a first end (66) of the hollow thread body (62) from outside of the hollow thread body (62), extends helically, enters the lumen of the hollow thread body (62), extends along the inner lumen surface (64) of the hollow thread body (62), and ends at a second location. In one embodiment, one end of the thread (68) is at the outside of the hollow thread body (62) at its first end (66), and the other end of the thread (68) is at the inside of the hollow thread body (62). The end of the thread (68) outside of the hollow thread body (62) forms a corresponding surface (70a), as shown in FIG. 3.

According to one embodiment of the present teaching, the corresponding surface (70a, 70b) is configured to make contact with the interface (50a, 50b) when an internal thread portion and an external thread portion are threaded to a pre-determined extent, thereby stopping further advancement of the threads (48, 68). In one embodiment, the corresponding surface (70a, 70b) is configured to match the interface (50a, 50b). In another embodiment, the corresponding surface (70a, 70b) does not match the surface profile of the interface (50a, 50b), but merely function to abut the interface (50a, 50b).

According to one embodiment of the present teachings, the corresponding, surface (70a, 70b) on the internal threaded portion (60) is a straight surface, for example, to match a straight interface (50a) on the external threaded portion (40a). In another embodiment, the corresponding surface (70a, 70b) on the internal threaded portion (60) is a curved surface, for example, to match a curved interface (50b) on the external threaded portion (40b). Similar to the interface (50a, 50b) on the external threaded portion (40a, 40b), according to one embodiment of the present teachings, the corresponding surface (70a, 70b) on the internal threaded portion (60) aligns with the longitudinal axis of the thread body (62), for example, as illustrated in FIG. 3, for matching the interface (50a, 50b) on the external threaded portion (40a, 40b) as shown in FIGS. 2A-2B. In another embodiment of the present teachings, the corresponding surface (70a, 70b) on the internal threaded portion (60) angles to the longitudinal axis of the thread body (62), for example, to match an angled interface (50a, 50b) on the external threaded portion (40a, 40b).

In one embodiment, the inner lumen of the hollow thread body (62) of the internal threaded portion (60) has a generally cylindrical shape, for example, as illustrated in FIG. 3, for matching the generally cylindrical shape of an external threaded portion (40a) as shown in FIG. 2A. In another embodiment, the inner lumen of the hollow thread body (62) of the internal threaded portion (60) has a generally conical shape, for example, to match a generally conical shape of the external threaded portion (40b). In another embodiment, the external profile of the internal threaded portion (60) can be other shapes or forms, for example, to match the corresponding shape or form of an external threaded portion (40b).

According to one embodiment of the present teachings, the thread (68) of an internal threaded portion (60) is configured to match the corresponding thread (48) of an external threaded portion (40). For example, the internal threaded portion (60) can have a right-handed thread that matches the corresponding thread (48) of the external threaded portion (40); or the internal threaded portion (60) can have a left-handed thread that matches the corresponding thread (48) of the external threaded portion (40). In another embodiment, the cross-sectional shape of the thread (68) of the internal threaded portion (60) is square, triangular, trapezoidal, or other shapes that matches the corresponding thread (48) of an external threaded portion (40). In another embodiment, the thread (68) of the internal threaded portion (60) has an angle that matches the corresponding thread (48) of an external threaded portion (40). In one embodiment, the internal threaded portion (60) is a single-start, for example, to match the corresponding external threaded portion (40). In another embodiment, the internal threaded portion (60) is a double-start, for example, to match the corresponding external threaded portion (40).

FIGS. 4A-4B illustrate embodiments of the present teachings where an internal threaded portion (60) and an external threaded portion (40) are assembled. In this embodiment, as the internal (60) and external (40) threaded portions are threaded together, the first end (66) of the internal threaded portion (60) is advanced toward the enlarged end portion (46) of the external threaded portion (40). When fully assembled, the interface (50) on the external threaded portion (40) contacts with the corresponding surface (70) on the internal threaded portion (60). The contact of the interface (50) and the corresponding surface (70) prevents the internal (60) and external threaded portions (40) from further rotating relative to each other.

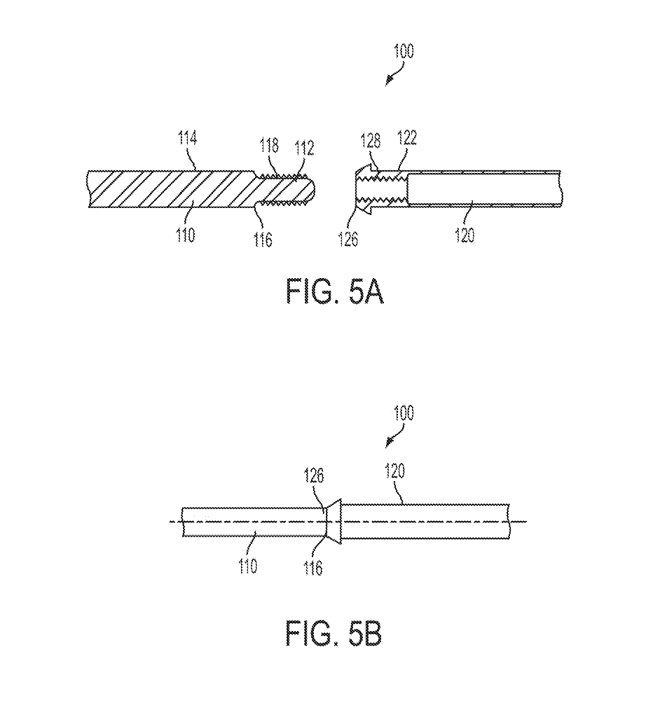

One skilled in the art understands that the torque strength of a traditional thread assembly gradually increases as the threads rotation angle increases. Upon reaching as certain degree, the torque strength of the thread assembly increases sharply. For a traditional thread assembly (100), as shown in FIG. 5A, the external threaded portion (110) has a thread body (112) with an enlarged end portion (114). The enlarged end portion 1114) of the external threaded portion (110) does not have an interface that prevents the internal threaded portion (120) from further advancing over the external threaded portion (110). The enlarged end portion (112) of the external threaded portion (110) has an under-head surface (116). As the threads (118, 128) of the internal (120) and external (110) threaded portions fully engage with each other, as illustrated in FIG. 5B, the under-head surface (114) of the enlarged end portion (116) of the external threaded portion (110) contacts the end surface (126) of the internal threaded portion (120). Because the threads of the internal (120) and external (110) threaded portions can still rotate relative to each other, the under-head surface (116) of the external threaded portion (110) and the end surface (126) of the internal threaded portion (120) can continue compressing each other.

The friction between these two surfaces (116, 126), namely, the under-head surface (116) of the external threaded portion (110) and the end surface (126) of the internal threaded portion (120), can absorb 50% or more of the total torque strength. When releasing the thread assembly (100) is attempted, the friction between these two surfaces (116, 126) can hinder the disengagement of the threads (118, 128).

According to one embodiment of the present teachings, for an anti-lockup thread assembly (30), the torque strength reaches a pre-set level and is prevented from increasing significantly beyond such pre-set level. As the internal and external threads (48, 68) fully engage with each other, as illustrated in FIGS. 4A-4B, the anti-lockup feature of the thread assembly (30), i.e. the interface (50) on the external threaded portion (40) and the corresponding surface (70) on the internal threaded portion (60), prevents the threads (48, 68) from further advancing over each other and the under-head surface of the enlarged end portion (46) of the external threaded portion (40) and the end surface of the internal threaded portion (60) from being further compressed to each other beyond a pre-designed level. As a result, the torque strength of the thread assembly will not increase significantly. Without being limited to any specific theory or hypothesis, without the added friction force between the two surfaces normally seen in traditional threaded assemblies, releasing such anti-lockup screw assembly becomes predictable.

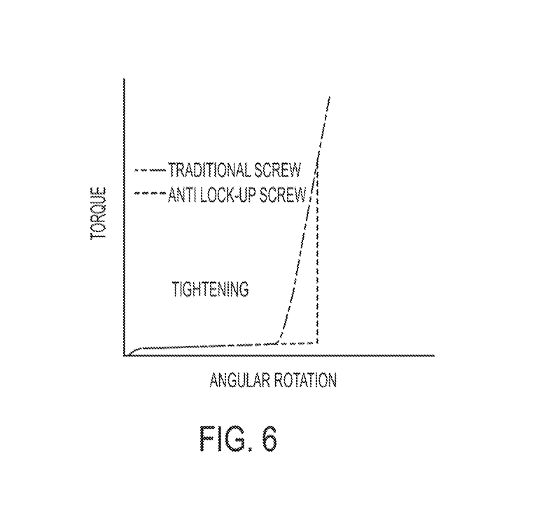

FIG. 6 illustrates torque-angular rotation curves showing the difference between a traditional thread assembly (100) and an anti-lockup thread assembly (30) according to an embodiment of the present teachings. As illustrated in FIG. 6, as a traditional thread assembly (100) being fully engaged, the angular rotation keeps increasing. Accordingly, the torque strength keeps increasing. As illustrated in FIG. 6, as an anti-lockup thread assembly (30) being fully engaged, the angular rotation discontinues. Consequently, the torque strength remains at its pre-designed level. One skilled in the art would understand that FIG. 6 is merely a schematic illustration aiming to exemplify difference between a traditional thread assembly and an anti-lockup thread assembly. This illustration is not to be used to limit the scope of the present teachings.

According to one embodiment of the present teachings, for a thread assembly (30) with anti-lockup feature, the torque strength to be overcome for releasing the external (40) and internal threaded portions (60) from each other are pre-determined by the design of the interface (50) and corresponding surface (70) and by the assembly process. Thus, after the thread assembly (30) is fully engaged, the torque strength is fully set. In various embodiments, with the anti-lockup design in place, releasing of such thread assembly (30) is predictable. To release the thread assembly (30), an initial releasing torque is used to overcome the friction between the under-head surface of the external threaded portion (40) and the end surface of the internal threaded portion (60) and disengage the external threaded portion (40) from the internal threaded portion (60). After that, the torque required to further unthread the assembly (30) is low. Upon further unthreading, the internal (60) and external (40) threaded portion (40)s are fully released from each other.

According to one embodiment of the present teachings, when the internal threaded portion (60) fully engages to the external threaded portion (40), the maximum torque strength of the thread assembly (30) is pre-set. In one embodiment, the torque is determined by the friction between the engaged threads and not subject to the additional under-bead friction. According to another embodiment, it takes 2-15 turns for the external (40) and internal (60) threaded portions to fully engage with each other and to reach the pre-set torque strength.

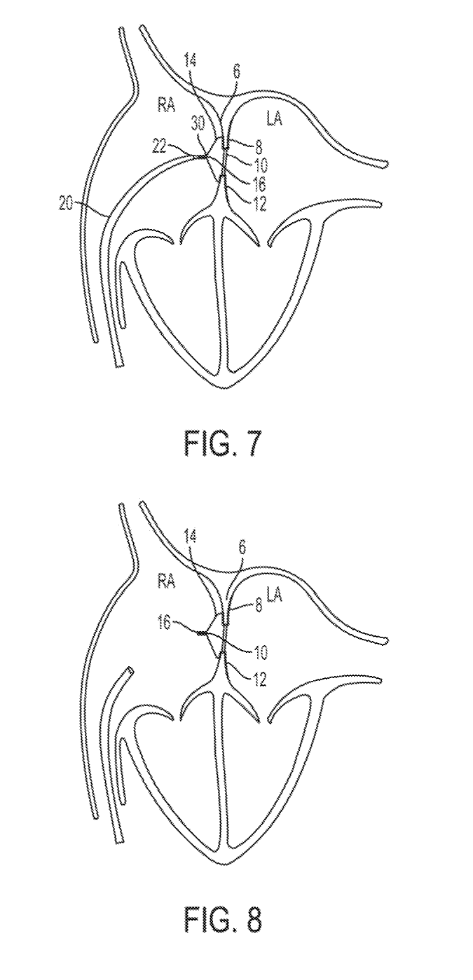

As described later in the present teachings, the exemplary thread assembly mechanism (30) with an anti-lockup feature is used for engaging a cardiac implant (10) to a delivery catheter (20). According to one embodiment of the present teachings, as shown in FIGS. 7-8, the delivery catheter (20) has a proximal end (not shown) and a distal end (22) with an exemplary external threaded portion (40) at the distal end (22) of the delivery catheter (20). The exemplary cardiac implant (10) has an elongated delivery profile and an expanded profile. The exemplary cardiac implant (10) further includes a proximal portion (14) and a distal portion (12). A proximal end (16) of the exemplary cardiac implant (10) includes an internal threaded portion (60) for engaging the external threaded portion (40) on the distal end (22) of the delivery catheter (20). Alternatively, the delivery catheter (20) can have an internal threaded portion (60) at its distal end (22), while the proximal end (16) of the implant (10) can have a corresponding external threaded portion (40). One skilled in the art would understand that the embodiments described in the present teachings should not be construed as limiting the present teachings.

According to one embodiment, medical devices incorporating inventions described in the present teachings have some similarities to those disclosed in U.S. Pat. Nos. 8,157,860; 8,172,896, and 8,252,042, all of which were filed on Mar. 8, 2010, and are entitled “Devices, systems and methods to treat heart failure,” and U.S. patent application Ser. No. 13/838,192, filed on Mar. 15, 2013, and entitled “Devices and Methods for Retrievable Intra-atrial Implants;” each of which is incorporated herein by reference in its entirety. Though not shown in the exemplary figures, one skilled in the art would understand that implants with other shapes, other configurations, and for other purposes can also incorporate inventions of the present teachings and be delivered percutaneously by a catheter.

FIG. 7 illustrates an embodiment of a cardiac implant (10) in its deployed configuration. The implant (10) includes an expanded distal flange portion, a core segment, an expanded proximal flange portion, and a proximal hub. The distal flange portion of the cardiac implant (10) is configured to oppose the septum (6) on the left atrial side. A proximal end of the distal flange connects to the core segment. The core segment of the cardiac implant (10) is configured to be placed through the aperture (8) in the septum (6). The core segment of the cardiac implant (10) connects the distal and proximal flanges. The proximal flange is configured to oppose the septum (6) on the right trial side. A distal end of the proximal flange connects to the core segment. A proximal end of the proximal flange connects to the proximal hub. The proximal hub allows the device to be connected to a delivery catheter (20).

According to one embodiment of the present teachings, similar to the embodiment described in FIG. 3, the proximal hub has a lumen with threads around the inner surface of the lumen in the form of a helix, forming an internal threaded portion. Such internal threaded portion is configured to engage a matching external threaded portion of the thread attachment mechanism at a distal end of a delivery catheter as described herein. In one embodiment, the internal thread starts from a first location on the proximal hub, extends helically along the inner lumen surface of the proximal hub, and ends at or near the second end of the proximal hub. In one embodiment, the internal thread remains inside the lumen. In one embodiment, the proximal hub also includes a corresponding surface at the proximal end of the proximal end. In one embodiment, the proximal hub remains outside of the hub lumen. The corresponding surface is configured to match an interface on the external threaded portion at the distal end of the delivery catheter. According to one embodiment, and similar to what has been described above, the corresponding surface could be a separate structure at the proximal end of the hub. According to another embodiment, and similar to what has been described above, the corresponding surface could be a part of the internal thread.

FIG. 7 further illustrates an embodiment of the delivery catheter (20) for engaging the cardiac implant (10) described above. According to one embodiment of the present teachings, the delivery catheter (20) includes a proximal end (not shown), a distal end (22), and an elongated body extending between the two ends. The distal end (22) of the delivery catheter (20) includes an external threaded portion. Similar to the embodiment described in relation to FIG. 2A, such external threaded portion has a thread body with threads around the thread body in the form of a helix and an enlarged proximal end. The thread starts from a distal end of the thread body, extends helically around the thread body toward the enlarged proximal end, and ends at an interface. Such interface is configured to match the corresponding surface at the proximal end of the thread on the proximal hub of a cardiac implant (10). According to one embodiment, and similar to what has been described above the interface could be a separate structure or part of the enlarged end of the thread body.

According to one embodiment, the external threaded portion at the distal end (22) of a delivery catheter (20) is configured to engage the internal threaded portion at the proximal end (16) of a cardiac implant (10). Upon engaging the implant (10) to the delivery catheter (20), the catheter-implant assembly is delivered percutaneously into the heart. In one embodiment, the delivery catheter (20) engaging the cardiac implant (10) is advanced over a guide wire placed across the atrial septum (6) beforehand. In another embodiment, the delivery catheter (20) engaging the cardiac implant (10) is advanced through a delivery sheath placed across the atrial septum (6) beforehand. In another embodiment, a delivery catheter (20) engaging the cardiac implant (10) is advanced directly through the blood vessel into the right atrium and across an aperture in the atrial septum (6), entering the left atrium.

According to various embodiments of the present teachings, the anti-lockup feature of the thread assembly, namely the corresponding surface on the internal threaded portion at the proximal end of an implant and the interface on the external threaded portion at the distal end of a delivery catheter, prevents the implant from being over-tightened to the delivery catheter. Thus, as the implant and delivery catheter are fully engaged with each other, the torque required to disengage the implant from the catheter in later release is pre-set. According to one embodiment, the control handle of the delivery catheter is configured to handle such torque strength for disengaging the implant from the delivery catheter.

FIG. 7 illustrates an embodiment of the present teachings where a cardiac implant (10) is deployed across the atrial septum. As shown in FIG. 7, the distal flange of the cardiac implant (10) is located near or against the left atrial side of the septum (6); the proximal flange of the cardiac implant (10) is located near or against the right atrial side of the septum (6); and the core segment of the cardiac implant (10) is positioned through an aperture in the septum (6). As illustrated in FIG. 7, the proximal hub of the cardiac implant (10) engages the distal end (22) of the delivery catheter (20) by the connection between the internal and external threaded portions of the thread attachment mechanism.

According to various embodiments of the present teachings, after a cardiac implant (10) is delivered to a treatment location, a clinician evaluates the deployment of the cardiac implant (10). If the deployment is deemed unsatisfactory, a clinician retracts the delivery catheter (20) proximally, thereby retrieving the cardiac implant (10) from the body. If the deployment is deemed satisfactory, a clinician proceeds to release the implant (10).

According to one embodiment of the present teachings, to release the implant (10) from the delivery catheter (20), a clinician rotates the delivery catheter (20) to disengage the internal and external threaded portions of the thread attachment mechanism. As the proximal and distal flanges of the cardiac implant (10) opposed against the septal wall, the implant (10) is prevented from rotating along with the catheter (20). As a result, the external threaded portion at the distal end (22) of the delivery catheter (20) controlled by the handle at the proximal end of the delivery catheter (20) separates from the internal threaded portion at the proximal end (16) of the implant (10). Once the implant (10) completely disconnects from the delivery catheter (20), as shown in FIG. 8, a clinician retracts the delivery catheter (20) further.

Although the present teachings disclose the steps of delivery, deployment, and release of a cardiac implant across the atrial septum, one skilled the in art would understand that these specific steps are treatment or implant specific and thus subject to change. Thus, specific embodiments disclosed in the present teachings should not be construed as limiting.

According to one embodiment, the thread attachment mechanism disclosed herein is useful for engaging an implant with a delivery catheter for percutaneous delivery and deployment. Although an exemplary cardiac implant is used for describing the present teachings, one skilled in the art would recognize that the present teachings can be used to engage, deliver, and deploy other catheter-based minimally invasive medical implants, for example, septal closure, urinal, gastro-intestinal, vasculatural, or esophageal implants etc. According to another embodiment, the thread attachment mechanism is useful for engaging an implant made with pre-shaping, laser cutting, or braiding techniques. According to another embodiment, the thread attachment mechanism is useful for engaging an implant made with plastic or metal, including shape memory alloys such as nitinol. Accordingly, the steps of delivery, deployment, and release of an implant varies according to the treatment purpose, the construction, and material of an implant.

Various embodiments have been illustrated and described herein by way of examples, and one of ordinary skill in the art will appreciate that variations can be made without departing from the spirit and scope of the present teachings. The present teachings are capable of other embodiments or of being practiced or earned out in various other ways, for example, in combinations, all of which are within the scope of the present teachings and the appended claims, when applicable, explicitly or under the doctrine of equivalents. Also, it is to be understood that the phraseology and terminology employed herein is for the purpose of description and should not be construed as limiting.

Unless otherwise defined, all technical and scientific terms used herein have the same meaning as commonly understood by one of ordinary skill in the art to which this present teachings belong. Methods and materials similar or equivalent to those described herein can be used in the practice or testing of the present teachings and are within the scope of the present teachings and appended claims when applicable. In case of conflict, the patent specification, including definitions, will control. In addition, the materials, methods, and examples are illustrative only and not intended to be limiting.

We claim:

1. A device comprising a medical implant and a delivery catheter for percutaneous delivery of the medical implant into a patient wherein the medical implant and the delivery catheter are connected by a thread attachment mechanism comprising:
an external threaded portion comprising a longitudinal axis and an interface, wherein at least one angle between the interface and the longitudinal axis of the external threaded portion ranges from 0° to about 70°; and
an internal threaded portion comprising a surface wherein the surface comprises a matching profile with the interface of the external threaded portion;
wherein the external and internal threaded portions are configured to engage each other.

2. The device of claim 1, wherein at least one angle between the interface and the longitudinal axis of the external threaded portion ranges from 0° to about 45°.

3. The device of claim 1, wherein at least one angle between the interface and the longitudinal axis of the external threaded portion ranges from 0° to about 30°.

4. The device of claim 1, wherein at least one angle between the interface and the longitudinal axis of the external threaded portion ranges from 0° to about 15°.

5. The device of claim 1, wherein at least one angle between the interface and the longitudinal axis of the external threaded portion ranges from 0° to about 5°.

6. The device of claim 1, wherein the external threaded portion comprises at thread body and a first thread, wherein the first thread extends around the thread body in a helical manner from a first end of the thread body to the interface.

7. The device of claim 1, wherein the external threaded portion comprises an enlarged end portion, wherein the interface is between the thread body and the enlarged end portion.

8. The device of claim 1, wherein the internal threaded portion comprises a hollow thread body and a second thread, wherein the hollow thread body comprises an inner lumen, the second thread extends along the inner lumen of the hollow thread body from the outside of the lumen at a first end of the hollow thread body to a second end inside the lumen, and the surface is at one end of the second thread.

9. A catheter for percutaneous delivery of a medical implant into a patient comprising a threaded portion at a distal end of the delivery catheter, wherein the threaded portion comprises a thread body comprising a longitudinal axis, an enlarged end portion, and an interface, wherein the interface is between the thread body and the enlarged end portion and at least one angle between the interface and the longitudinal axis of the thread body ranges from 0° to about 70°.

10. The catheter of claim 9, wherein at least one angle between the interface and the longitudinal axis of the thread body ranges from 0° to about 45°.

11. The catheter of claim 9, wherein at least one angle between the interface and the longitudinal axis of the thread body ranges from 0° to about 30°.

12. The catheter of claim 9, wherein at least one angle between the interface and the longitudinal axis of the thread body ranges from 0° to about 15°.

13. The catheter of claim 9, wherein at least one angle between the interface and the longitudinal axis of the thread body ranges from 0° to about 5°.

14. A thread attachment mechanism connecting a medical implant and a delivery catheter for percutaneous delivery of the medical implant wherein the thread attachment mechanism comprises an external threaded portion, an internal threaded portion, and an anti-lockup feature.

15. The thread attachment mechanism of claim 14, wherein the external threaded portion comprises a longitudinal axis and an interface at one end of the external threaded portion and at least one angle between the interface and the longitudinal axis ranges front 0° to about 70°.

16. The thread attachment mechanism of claim 15, wherein the at least one angle between the interface and the longitudinal axis ranges from 0° to about 45°.

17. The thread attachment mechanism of claim 15, wherein the at least one angle between the interface and the longitudinal axis ranges from 0° to about 30°.

18. The thread attachment mechanism of claim 14, wherein the internal threaded portion comprises a longitudinal axis and a surface at one end of the internal threaded portion and at least one angle between the surface and the longitudinal axis ranges from 0° to about 70°.

19. The thread attachment mechanism of claim 18, wherein the at least one angle between the surface and the longitudinal axis ranges from 0° to about 45°.

20. The thread attachment mechanism of claim 18, wherein the at least one angle between the surface and the longitudinal axis ranges from 0° to about 30°.
